(19) United States
(12) Patent Application Publication
(10) Pub. No.: US 2025/0319880 A1
(43) Pub. Date: Oct. 16, 2025

(54) HORIZONTAL GAZE NYSTAGMUS TRANSMISSION INTERLOCK SYSTEM AND METHOD

(71) Applicant: James Madison University, Harrisonburg, VA (US)

(72) Inventors: Ahmad Salman, Harrisonburg, VA (US); Roderick H. MacDonald, Staunton, VA (US); Chase Coleman, McLean, VA (US); Matthew Jenkins, Harrisonburg, VA (US); Patrick Dodds, Harrisonburg, VA (US); William Roberts, Buena Vista, VA (US); Abdullah Alghoniemy, Harrisonburg, VA (US)

(73) Assignee: James Madison University, Harrisonburg, VA (US)

(21) Appl. No.: 19/175,284

(22) Filed: Apr. 10, 2025

Related U.S. Application Data
(60) Provisional application No. 63/632,711, filed on Apr. 11, 2024.
(60) Provisional application No. 63/632,716, filed on Apr. 11, 2024.

(51) Int. Cl. B60W 40/08; B60K 28/06; B60W 10/10; B60W 10/18; G06F 3/16; G06F 21/60; G06T 7/11; G06V 40/16; G06V 40/18

(52) U.S. Cl. CPC B60W 40/08; B60K 28/063; B60W 10/10; B60W 10/18; G06F 3/167; G06F 21/602; G06T 7/11; G06V 40/168; G06V 40/18; B60W 2040/0818; B60W 2040/0872; B60W 2540/221

(57) ABSTRACT

A device, system and methodology for horizontal gaze nystagmus (HGN) testing. Prior to a test subject taking an action that is verboten in an impaired state, such as driving a vehicle or operating complex or dangerous machinery, the test subject is positioned within a face recognition box of a screen and a HGN simulation test performed, capturing HGN eye movements of the test subject from which the present HGN physiological state of the test subject is determined. The present HGN physiological state of the test subject is compared to a reference HGN state to determine whether the test subject is impaired. Impairment causes temporary restriction of the functionality of the machinery or vehicle.

### PRIORITY CLAIM

This application claims the benefit of U.S. Provisional Patent Application Nos. 63/632,711 (filed on Apr. 11, 2024) and 63/632,716 (filed on Apr. 11, 2024), the contents of which are both incorporated herein by reference in their entireties.

BACKGROUND

Drinking and driving has been and continues to be a serious problem. In 2022, there were more than 13,524 deaths from alcohol related crashes in the United States (National Center for Statistics and Analysis, 2024 May). Great strides have been made since the 1980s when Mothers Against Drunk Driving (MADD) started advocating for stricter laws. Drinking and driving was criminalized and laws were passed based on a deterrent model of swift, certain, and severe punishment. The number of alcohol related traffic fatalities decreased substantially from the 1980s to the late 1990s and then became constant with 20 percent of all traffic fatalities caused by drivers with a blood alcohol content of 0.08 or above. Behavioral changes have had a significant impact on reducing the incidence of drinking and driving. To further reduce or eliminate drinking and driving a technological solution is needed.

As people have been returning to the streets after COVID, so too have the crashes involving them, both fatal and otherwise. According to the National Highway Traffic Safety Administration's most recent publication, total traffic accidents have risen by 10% in the time between 2020 and 2021, accounting for 42,939 deaths on the road. Of these deaths, alcohol use was involved in 13,384, nearly one third of the total in 2021 and a 14% rise from the previous year's findings. Of these alcohol impaired fatalities, closed air vehicles account for the vast majority, with less than 11% of fatalities coming from motorcycles.

It is no surprise, then, that many attempts have been made to solve such a problem, though many have been reactionary. As stated in a journal from the National Library of Medicine, in 2011 all fifty states passed sanctions specific to those who have repeated driving while under the influence convictions on their record, and 38 states allow sobriety checkpoints. On the other hand, interlock systems have been underutilized in legislation until recently, with only 9 states requiring their installment for DWI offenders. All of these penalties, however, rely on the metric of Blood Alcohol Content (BAC), a way in which to quantify and measure the level to which someone is alcohol impaired in use since 1938 that tests the amount of alcohol in a person's blood.

Breathalyzers have been available since the 1950s and for the last couple of decades have been available as hand-held devices and as ignition interlocks in cars. These devices require the driver to breathe into the device and it then measures the driver's blood alcohol and can prevent the car from starting when the blood alcohol content is too high. When installed in vehicles the breathalyzer is a deterrent, but it will also pick up alcohol in the air from other passengers. It also fails in cold conditions as moisture in one's breath will crystallize and freeze.

Horizontal gaze nystagmus (HGN) is an involuntary eye movement that can be caused by intoxication with alcohol or drugs or other conditions indicating impairment. It is one of three field sobriety tests that police officers use to determine if a driver is impaired. HGN is not always present in intoxicated drivers, but it is a very sensitive indicator of intoxication or impairment when it is present.

There is a need for HGN field sobriety test devices and testing in vehicles and other machines that can help improve the accuracy of law enforcement's ability to identify impaired drivers. Currently, HGN tests are performed manually by police officers, which can be subjective and prone to error. HGN devices can provide objective and accurate results, which can help to ensure that impaired drivers are taken off the road and prevented from operating machinery that requires a sober status.

### BRIEF DESCRIPTION OF THE DRAWINGS

The accompanying drawings provide visual representations which will be used to describe various representative embodiments more fully and can be used by those skilled in the art to better understand the representative embodiments disclosed and their inherent advantages. In these drawings, like reference numerals identify corresponding or analogous elements.

FIG. 1 is a flowchart of a method of HGN simulation testing, in accordance with embodiments of the disclosure.

FIG. 2 is a system block diagram of a HGN simulation testing system, in accordance with embodiments of the disclosure.

FIG. 3 is a functional block diagram of a HGN simulation testing system, in accordance with embodiments of the disclosure.

FIG. 4A shows an example of a two guiding squares seen by the user when too far away from the screen, during positioning of a user for a HGN simulation test, in accordance with embodiments of the disclosure.

FIG. 4B illustrates a single guiding square shown the user in the GUI when too close to the screen, during positioning of a user for a HGN simulation test, in accordance with embodiments of the disclosure.

FIG. 4C illustrates two guiding squares, which may be of different colors, to communicate when the user is not centered, during positioning of a user for a HGN simulation test, in accordance with embodiments of the disclosure.

FIGS. 5, 6 and 7 illustrate pretest face positioning during a HGN simulation test,, in accordance with embodiments of the disclosure.

FIG. 8 illustrates a screen with a simulation test interface, in accordance with embodiments of the disclosure.

FIGS. 9A, 9B, 9C illustrate different dot positions during a HGN simulation test using dots, in accordance with embodiments of the disclosure.

FIGS. 10A, 10B, 10C illustrate the results of analysis performed by an eye gaze tracking analysis module of a controller during a failed HGN simulation test, in accordance with embodiments of the disclosure.

FIGS. 11A, 11B, 11C illustrate the results of analysis performed by an eye gaze tracking analysis module of a controller during a passed HGN simulation test, in accordance with embodiments of the disclosure.

FIGS. 12A, 12B, 12C illustrate example baseline test scores for different users for different types of baseline testing, in accordance with embodiments of the disclosure.

FIG. 13 illustrates a HGN simulation test flow, in accordance with embodiments of the disclosure.

FIGS. 14A, 14B illustrate accuracy using a Random Forest N Estimator and for various types of testing across different users, in accordance with embodiments of the disclosure.

FIGS. 15A, 15B, 15C illustrate test graphs generated after different types of HGN simulation testing, in accordance with embodiments of the disclosure.

FIG. 16 is a model confusion matrix, in accordance with embodiments of the disclosure.

FIG. 17 is a flowchart illustrating HGN data processing, in accordance with embodiments of the disclosure.

FIG. 18 is a functional block diagram of a HGN simulation testing system providing for modification of vehicle or machine operation, in accordance with embodiments of the disclosure.

FIG. 19 is a functional block diagram of a HGN simulation testing system providing for modification of vehicle or machine operation, in particular for disabling the shift lock solenoid of a vehicle, in accordance with embodiments of the disclosure.

FIG. 20 is a block diagram illustrating an immobilization mechanism for a vehicle or machine, in accordance with embodiments of the disclosure.

### DETAILED DESCRIPTION

After the lifting of Prohibition, nearly all states established a drinking age in the U.S. of 21 years old in order to restrict youth access to alcohol. In the 1970s, however, many states chose to lower their minimum legal drinking age. This resulted in a dramatic increase in alcohol-related crashes, as well as other drinking-related deaths. A backlash followed and states began passing laws aimed at reducing the incidence of drinking and driving. The legal age for drinking was increased to 21 in all states by 1988, 40 states implemented mandatory license suspensions for DWI offenses, zero tolerance for underage drinking and driving became the law in all states by 1998, and legal blood alcohol content for conviction was reduced from 0.10 to 0.08 in all states by 2005. Mothers Against Drunk Driving (MADD) is an advocacy non-profit that was founded in 1980, with the goal of educating and protecting the public from drinking drivers. Since its founding, alcohol-related deaths have decreased by more than 50 percent, and according to MADD, more than 370,000 lives have been saved. In the past twenty years, however, fatal crashes as a result of drinking drivers has plateaued, implying that social and legal interventions are no longer resulting in behavioral changes. A more innovative solution is needed to address the problem and further decrease the number of deaths related to drinking and driving.

Over the years there have been a variety of different tests employed by law enforcement in the field to identify drivers potentially under the influence of alcohol. They are known as field sobriety tests. These tests are unable to provide an exact reading, but instead a set of physical and mental tasks that the person in question must complete to prove that they are not impaired or intoxicated. There are three main field sobriety tests: the one-leg stand test, the walk-and-turn test, and the horizontal gaze nystagmus test. Failure of these tests provides officers with probable cause to detain suspects and offer a breathalyzer test in the field. Refusal will lead to automatic license suspension in most states. Failure of a breathalyzer in the field will result in an arrest and testing at the police station with a calibrated breathalyzer. A BAC of 0.08 or above is a failure and is considered per se evidence of driving under the influence. In contrast, the methods, devices and systems described herein avoid this by preventing the operation of a vehicle or machinery while impaired. In addition, the device encrypts all of the data collected by a HGN testing system, making it private to the user only. These field sobriety tests conducted at impairment checkpoints are also only effective if the person is caught in time by the officer, and many times they are not. The technology system and device described herein takes a proactive approach to public safety, as opposed to the reactive approach of using law enforcement.

Law enforcement officers commonly use field sobriety tests, such as the Horizontal Gaze Nystagmus (HGN) to determine probable cause for an arrest and then testing with a breathalyzer to determine the blood alcohol content of the driver. HGN measures the physiological response in the eye that is present when under the influence of a substance. Using ocular-graphic techniques, the type of nystagmus being exhibited, whether it be pendular (sinusoidal) or jerk (fast, sudden corrections), can be determined. The latter of these is more common in people who are under the influence of alcohol, known as vestibular nystagmus.

Horizontal Gaze Nystagmus (HGN) is used as a method of impairment testing, in which a police officer may quickly test a person's impairment by examining the movement of their eyes when tracking an object. In this disclosure, the refinement and modulization of a proactive sobriety test based on current law enforcement's HGN test is presented. Using this test, the HGN testing device and system is capable of sensing, testing, and determining personalized conclusions on a user's level of intoxication or impairment based on the nystagmus (erratic eye movements) they may or may not exhibit.

The technology presented herein presents a unique and distinct approach in impairment detection, making use of HGN, a method of testing for intoxication, drug use or other impairment by examining the reactions of a test subject's eyes while tracking an object. To further enhance the technology, a personalized classification model may be used in data processing. To that end, machine learning algorithms such as Random Forest (RF) or Siamese neural networks may be used in data processing.

HGN is used by law enforcement as a probable cause assessment test after a suspect has been pulled over, and as of 2021, can be used as scientific or characteristic evidence in U.S. courts. In contrast to the legal system's use of HGN to convey probable cause, an improved method, device and system of HGN testing is administered before a vehicle is on the road, using the HGN test as a prevention mechanism rather than as a punitive means. The Random Forest (RF) method involves the creation of many different and randomized decision trees, and then the collection of individual results into a single decision, hence the “forest.” Researchers have used RF in Driving While Intoxicated (DWI) prevention to analyze the effectiveness and placements of random BAC tests and it has proven to be an effective way to process data. A meaningful advantage of RF is its ability to not only compare results to a baseline, but also compare similarity between results. This capability is important when working with individuals' appearances and facial features because in accordance with certain embodiments of the invention, each individual may have their own baseline results, meaning an effective machine learning algorithm is able to adjust to each person's personalized baseline. RF models also solve the issue of overfitting, as a single decision tree can be over tuned to training data, making it ineffective when put in practice.

As described herein, a HGN testing device or system can be inexpensively installed in machines and vehicles and is capable of detecting impairment reflected by a failing HGN test in a matter of seconds, temporarily immobilizing the machine or vehicle, and preventing a driver from operating a machine or driving a vehicle. The HGN device or system encrypts information to prevent access to failed tests by others.

Driving while intoxicated or impaired continues to be a morbid issue in the United States, responsible for causing approximately one-third of all fatal car crashes, claiming over 11,000 victims each year. Psychological studies have shown that those who drive under the influence are likely to be repeat offenders. An objective is to remove human error by building a technological solution to address the needs specified by the Department of Transportation. While incorporating physiological analysis to determine sobriety based upon a passive HGN test, if an individual is attempting to drive while intoxicated or impaired, a personalized machine-learning algorithm may be calibrated to said individual to test their sobriety while protecting their privacy. The result of the sobriety test will determine if the individual is able to operate the vehicle, immobilizing the vehicle temporarily, if the driver is intoxicated or impaired. The HGN methodology described herein can identify whether or not a driver is impaired with a clear distinction in a very short amount of time without compromising the user's privacy.

An in-vehicle HGN field sobriety test device and system that disables the driver's ability to mobilize the vehicle, such as disabling the vehicle's ability to shift into gear if the driver is deemed intoxicated or impaired, is presented. This is a valuable tool not only for consumers but for insurers looking to assess driver risk by incorporating such technology into the telematic discount programs. The technology can also benefit law enforcement, fleet management companies, and workplaces. In short, the technology presented herein can help improve the accuracy of impaired driver detection, prevent accidents, and save lives.

An important distinction between the improved technology described herein and existing HGN field sobriety tests is that the HGN simulation testing of the present disclosure includes taking action to prevent the impaired or intoxicated driver from operating a vehicle or other machine, whereas other approaches in the art serve only as evaluation or logging tools that notify the driver or third parties of the driver's unfit condition to drive, without physically disabling operation or mobilization of the vehicle or machine.

A few reasons why the HGN testing device, system and methodology of the present disclosure may be beneficial, include:
1) Impaired Driving Detection: HGN is one of the three standardized field sobriety tests recognized by the National Highway Traffic Safety Administration (NHTSA) in the United States. Law enforcement officers often use HGN tests to assess whether a driver is intoxicated or impaired. Having a device, system and methodology specifically designed for this purpose in vehicles can enable law enforcement to conveniently conduct HGN tests on the spot, potentially leading to more accurate detection of impaired drivers.
2) Enhanced Road Safety: Impaired driving is a significant contributor to traffic accidents and fatalities. By incorporating HGN field sobriety test devices and systems into vehicles, there is a potential to discourage individuals from driving under the influence. The presence of such devices and systems could serve as a deterrent and increase awareness about the risks of impaired driving, thereby promoting safer roads.
3) Real-Time Monitoring: While breathalyzers are commonly used to measure blood alcohol concentration (BAC), they do not provide real-time monitoring of a driver's current impairment level. HGN field sobriety test devices and systems as described herein, on the other hand, can detect the presence of nystagmus, indicating impairment at the time of the test. This real-time monitoring capability can provide valuable information to drivers, law enforcement, and other stakeholders.

A device, system and methodology for HGN testing of operators or a vehicle or a machine is presented, including an HGN field sobriety device or system, a machine or vehicle transmission system, and machine learning. While incorporating physiological analysis to determine sobriety based upon a passive HGN test, if an individual is attempting to drive while intoxicated or impaired, a personalized machine-learning algorithm may be calibrated to said individual to test their sobriety while protecting their privacy. The result of the sobriety test will determine if the individual is able to operate the machine or vehicle, immobilizing the machine or vehicle temporarily if the driver is intoxicated or otherwise impaired. Initial results show that this whether or not a driver is impaired can be identified with a clear distinction in a very short amount of time without compromising the user's privacy.

To elaborate, an HGN measures the physiological response in the eye that is present when under the influence of a substance. Using an ocular-graphic technique, the system can determine the type of nystagmus being exhibited, whether it be pendular (sinusoidal) or jerk (fast, sudden corrections). The latter of these is more common in people who are under the influence of alcohol, known as vestibular nystagmus. The HGN test described herein can be implemented by a device or system that can inexpensively be installed in vehicles or other machines and is capable of detecting impairment of a user in a matter of seconds, temporarily immobilizing the machine or vehicle, such as preventing a driver from driving a vehicle. The device, system and methods described herein encrypt information to prevent access to failed tests by others.

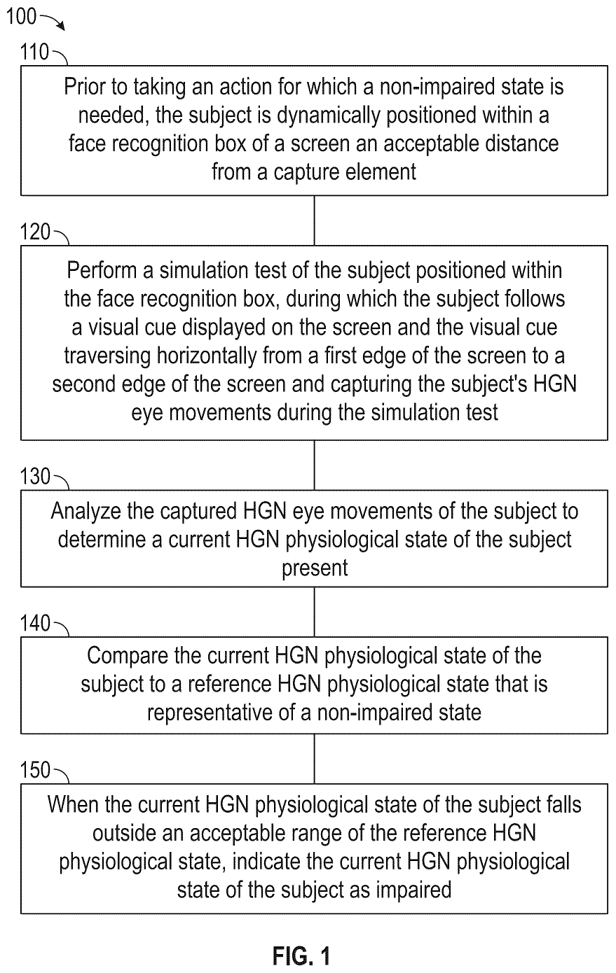

Therefore, in accordance the present disclosure, flow 100 of in FIG. 1 illustrates a methodology for performing horizontal gaze nystagmus (HGN) testing. While testing is described with respect to an operator/driver of a vehicle, the disclosure is not so limited. As previously described, the HGN testing methodology can be applied to a test subject or user of other types of machines, including but not limited to, operators of airplanes, remotely controlled machines, such as drones, boat or ship captains, operators of manufacturing equipment, operators of potentially dangerous equipment such as chainsaws, sanders, etc., traffic controllers, and other machinery requiring a non-impaired state of an operator/user. At 110, prior to taking an action for which a non-impaired state is needed, the subject is dynamically positioned within a face recognition box of a screen an acceptable distance from a capture element. The terms test subject, subject, user, participant, etc. of a HGN simulation test may be interchangeably used. At 120, a HGN simulation test of the test subject positioned within the face recognition box is performed, during which the test subject follows a visual cue displayed on the screen and the visual cue traversing horizontally from a first edge of the screen to a second edge of the screen and capturing the test subject's HGN eye movements during the simulation test. The screen is stationary during the HGN simulation test. At 130, the captured HGN eye movements of the test subject are analyzed to determine a current HGN physiological state of the test subject at the present time. At 140, the current HGN physiological state of the test subject is compared to a reference HGN physiological state that is representative of a non-impaired state. Finally, at 150, when the current HGN physiological state of the test subject falls outside an acceptable range of the reference HGN physiological state, the current HGN physiological state of the test subject as impaired is indicated.

As described herein and with regard to the reference HGN physiological state that is representative of a non-impaired state, the disclosure supports multiple machine learning-based approaches to detect impairment using horizontal gaze nystagmus (HGN), each addressing the problem from a distinct perspective. One approach involves training a generalized model on a broad dataset composed of gaze tracking test results collected from a diverse population. These test results are labeled according to the test subject's physiological condition during testing, distinguishing between unimpaired and impaired states. By learning from a wide range of gaze patterns across various users and conditions, this approach enables the system to identify common indicators of impairment based on generalized trends in eye movement behavior. A second approach focuses on individualized analysis by comparing a test subject's current gaze behavior to their previously recorded baseline data. This method learns the distinctions between normal and impaired states on a per-user basis by evaluating the similarity between gaze patterns from different sessions. During real-time operation, the system assesses how closely a new HGN simulation test aligns with the user's personalized, established non-impaired baseline. If a significant deviation is detected, it is used as an indicator of possible impairment. These complementary strategies enable both population-level classification and personalized detection, offering flexibility in deployment depending on context, available data, and desired specificity.

Returning again to Horizontal Gaze Nystagmus (HGN), HGN is a term used to describe repetitive oscillatory movement of the eye, that, based on frequency and amplitude, can be used to determine an individual's sobriety and/or impairment. As blood alcohol content increases, so does the alcohol concentration within other bodily fluids, including that which can be found in the inner ear. The inner ear system, or the vestibular system, acts as an accelerometer for the brain, helping to determine a body's orientation to or within the surrounding environment. While this function prevents getting dizzy on a day-to-day basis, those who consume enough alcohol will be familiar with the effects on equilibrium or coordination of the substance, causing balancing issues, and even escalating to nausea. These side effects are a direct result of the chemical change in this fluid. As a result, a person's eyes will react to their body's change in speed and direction with “jerking” eye movements, as the eyes search for objects to orient. The same phenomenon can be observed after spinning in circles.

The device and system herein are designed to measure this oscillation, which is known to occur and progressively increase in intensity beginning at a BAC of 0.05. Currently in the state of Virginia, for example, the BAC limit that is considered to be driving while intoxicated corresponds to a BAC of 0.08. When conducted in the field, police officers have approximately 70 percent accuracy with this test. This requires observing a frequency of 3-4 Hz and an amplitude of 2-3 degrees. The test, conducted 12 inches from the test subject's face, measures these parameters by graphing the gaze over time as a position function, and analyzing the reaction in the eyes at either horizontal extreme. This is because a vestibular nystagmus will present as jerking horizontal movements until the 45 degree point from the center line of the gaze, and as it is moved further to the corners of the eyes, the nystagmus will present itself as a vertical jerking.

### System Overview

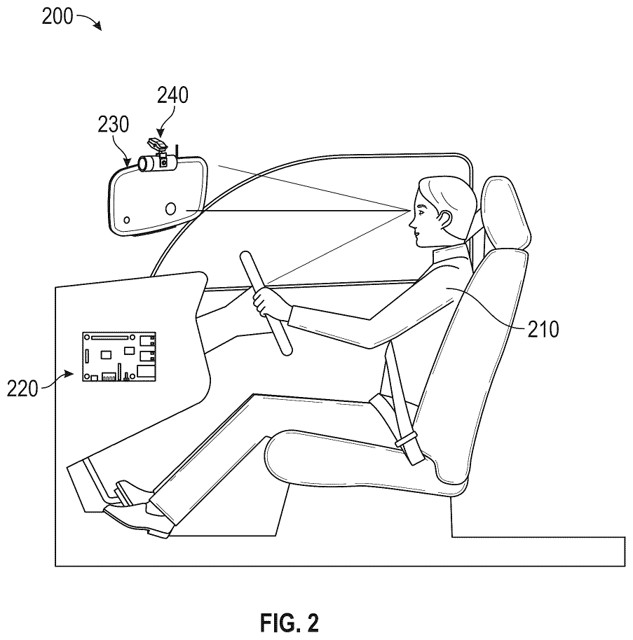

In accordance the present disclosure, system block diagram 200 of FIG. 2 illustrates an example embodiment for implementing horizontal gaze nystagmus (HGN) testing of a user, also referred to herein as a subject, test subject, participant, operator or the like. In the example embodiment shown in FIG. 2 the user 210 is seated within a seat of a vehicle. It is also envisioned that for other machines, which may or may not be vehicles, the user may not be seated but in a standing or even supine position so long has the user can see the screen 230 and have their eye(s) tracked by a capture element 240, such as a camera or other sensor(s) capable of tracking eye or head movement as shown. A controller, e.g. a microcontroller, which may be integrated in the machinery or vehicle though this is not required, is in communication with and controls operation of screen 230 and camera 240. The controller 220, screen 230 and capture element 240 may be integrated into the vehicle as shown, in which in this embodiment, the screen 230 and capture element 240 form part of a visor or a rear view mirror. Screen 230 may be a touch screen or the test subject may interface by a mouse or other means.

Screen 230 may be controlled by controller 220 and capture clement 240 could also be part of or attached to a heads-up display, a windshield, and/or a dashboard of the vehicle/machine. The design of the components of the HGN simulation system may be modular, allowing for them to be physically separable. For example, screen 230 and camera 240 may be modular or physically separable. Or, the test module of controller 220 may be housed separately, e.g., visor-mounted or console-integrated or wearable. Additionally, these elements, particularly the capture element and screen 230, may be added to the existing structure of the vehicle/machine, as might be the case where a screen separate from the vehicle is attached to a visor, a rearview mirror, heads-up display, a windshield, and/or a dashboard of the vehicle/machine. Such might be the case, for example, where the vehicle/machine is retrofitted with the HGN system described herein. Or, these components may be after-market functional upgrades to the vehicle/machine. Accordingly, as used herein the term “system” may be used interchangeably with the term “device.” It is understood and anticipated that the HGN simulation testing system may be implemented within a single device, such as a handheld device, laptop, testing device or the like, or the system components may be embodied in separate locations and within different hardware, all without straying from the intended scope described in the disclosure.

Functionally, screen 230 is configured to display a visual cue, such as a dot, as part of a HGN simulation test structured to elicit horizontal gaze tracking behavior of the user. In certain embodiments, screen 230 may be controlled by controller 220 to display a dot horizontal to the driver's gaze to conduct the HGN test. Capture element 240 is configured to record eye movement during the HGN test, such as in the form of a video of eye movement that is recorded and sent to the microcontroller/controller 220 for analysis. Controller 220 is used to analyze the recorded video and send the digital signal indicative of the test results to the machine/vehicle's control system to operate normally or to immobilize/suspend normal operation of the vehicle/machine, based upon the test results formed from analysis of the user's current physiological state.

In keeping with certain example embodiments, with regard to the hardware of the HGN testing system, the primary computing unit may be a Raspberry Pi (RPi) 4 Model B single-board computer running Raspbian. The RPi utilizes a 1.5 GHz quad-core Broadcom BCM2711 Cortex-A72 (ARM v8) 64-bit CPU, 8 GB LPDDR4-3200 SDRAM, and support for dual-band 802.11ac wireless networking, Bluetooth 5.0, and Gigabit Ethernet.

The screen 230 may be, for example, a 7-inch touch screen display coupled to the RPi's Display Serial Interface (DSI) connector in order to facilitate communication. The user may interact with the device by selecting options and entering data via a user interface, such as using a touch screen. As part of the HGN test, the test subject's eye movements are additionally recorded using a capture element 240, such as a RPi Camera Module V2 module that is connected to the RPi's Camera Serial Interface (CSI) port. The camera has an 8-megapixel sensor and supports 1080p30, 720p60, and VGA90 video modes, for example. This model provides high resolution video capture while keeping the system compact and is moreover also easy to mount within the vehicle system shown. Further, a 4-pin relay may be used, which is a simple relay with a solenoid and a switch, to control the opening and closing of the break-transmission circuit switch, explained below. The excess wires used may range from gauges: 18-22.

Consider the following example embodiment. Once the driver 210 takes their position and attempts to start the vehicle, the visor goes down revealing a camera 240 and an LED display screen 230. First the camera 240 sends a snap image of the driver to a Single Board Computer (SBC), i.e. microcontroller 220, which identifies the driver and loads their baseline data gathered during an initial one-time training phase. The baseline data is compared to the results of the HGN test to determine their sobriety. The test is performed by displaying a dot on an LED screen 230 attached to the car visor on the driver's side. The dot moves horizontally across the display screen 230 while the camera 240 records the eye movement during the HGN test. Once the test is completed, the recordings are sent to the SBC for analysis, with the SBC 220 issuing a signal to allow the transmission of the vehicle to function normally in case of a passed test or a signal to immobilize the vehicle, such as to lock the transmission, or to suspend normal operation of a machine, in the case of a failed test.

The HGN test is conducted by keeping an individual's head stationary, noting that for the average person the ability to smoothly track a visual cue or object, the object (dot) must move under 60 degrees per second. The average person has a field of view of 120 degrees. The object needs to pass through the field from one extreme to the next, with the goal being for the whole test and analysis to be conducted in under 10 seconds, for example.

Overall, the hardware arrangement may be made to be portable, small, and simple to put together, allowing for a wide range of applications for the HGN testing system/device.

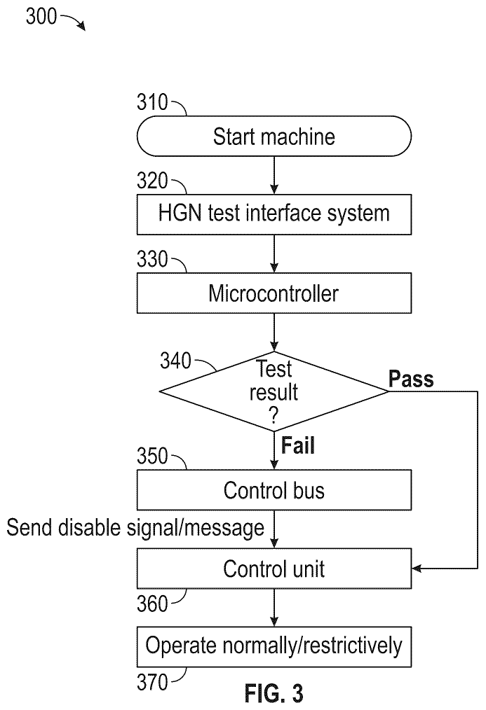

Referring now to FIG. 3, functional block diagram 300 illustrates an overall flow of an HGN simulation testing system for HGN testing an operator of a machine, which may be a vehicle, such as an automobile, a boat or ship, airplane, or a user operated machine, such as remotely controlled machines or drones, manufacturing equipment, potentially dangerous equipment such as chainsaws, sanders, etc., traffic controllers, and other machinery; such machinery calls for a non-impaired state of an operator/driver/user. At 310 the machine may be started; this is an optional step as perhaps the HGN testing system/device may be separate from a machine to be operated by the user or perhaps the user is not allowed to start the machine until successfully passing the HGN test. At 320, the user interfaces with a HGN test user interface that will ensure proper positioning of the user and successful completion of the HGN test. The captured readings of the HGN test, are submitted to a controller, such as a microcontroller, for analysis and generation of a test result indicative of the current physiological state of the user. The test result may be a score, as will be described, it may be a range or more qualitative data. At decision block 340, the generated test result is compared to a reference HGN physiological state by the analysis module of the microcontroller to determine whether the user passes or fails the HGN test. The reference HGN physiological state may be a personalized baseline HGN physiological state that is specific to the test subject and is determined during a training phase of a baseline simulation test that the user undergoes one time.

If the analysis by the analysis module of the controller 330 indicates that the user has a current physiological state that falls outside an acceptable range of the reference HGN physiological state and has thus failed the HGN test, then a control bus in communication with a control unit, such as an engine control unit (ECU) of a vehicle, boat, plane, drone or other machine, sends an indication of this failure to a control unit at 350. The indication may be a digital signal or a message that when received by the machine control unit at 360 causes it to suspend or prevent normal operation of the machine. The control unit will in accordance with the failure indication sent by control bus cause the machine to operate restrictively at 370. In the case of a vehicle, for example, preventing of normal operation of the vehicle may allow the vehicle to be started but not be mobilized due to the impaired state of the user. Or, the control unit of a machine may prevent the machine from being operated by the user altogether while the user is in an impaired state. If, conversely, the HGN simulation test indicates that the current state of the user of the machine is not impaired, i.e. the HGN simulation test result is pass, then normal operation of the machine is allowed. Depending on the type of machine and intended operation by the user when non-impaired, the user may be allowed to turn on the machine at different times, such as before the dynamic positioning portion of the setup for the HGN simulation test or before performing the HGN simulation test. In other examples, where no operation of the machine is allowed until the user successfully demonstrates a passing current HGN physiological state, the user may only be allowed to start the machine when the user's current HGN physiological state does not fall outside the acceptable range of the reference HGN physiological state.

It is further envisioned that there may be provided, in appropriate circumstances, a user override mode(s) for the machine or vehicle interlock (with notification) for emergency use, even if it is disabled by default due to an HGN impairment on the part of the operator/driver.

With regard to process of the HGN simulation test itself, there may be several portions of stages of the test that may be implemented using a combination of hardware, firmware and software and algorithm components, in cooperative arrangement. These several major stages each play a role in ensuring the accuracy and reliability of the final test results. These stages are pretest face positioning, identification, visual cue (dot) simulation, head movement detection, and eye gaze tracking, respectively.

Pretest Face Positioning: The first stage involves positioning the user's face in a precise and optimal manner for the testing process within a face recognition box of a graphical user interface. Before administering the HGN simulation test, it is important to ensure that the participant's face is correctly positioned within the camera frame to obtain accurate eye gaze data. To achieve this, the capabilities of the face-api JavaScript library, a powerful tool for face detection and recognition in real-time video streams, can be leveraged. Using face-api, the HGN testing system initiates a pre-test positioning verification process, in which the participant's face is continuously monitored in real-time as they approach the camera. Upon detecting the presence of a face within the camera frame, the face-api library assesses the positioning and orientation of the face relative to predefined guidelines for optimal test conditions.

Once the participant's face is detected, the system prompts them to adjust their position if necessary, providing visual feedback in real-time to guide them towards the correct alignment. This feedback may include on-screen instructions or visual cues overlaid on the video feed, directing the participant to center their face within the frame and maintain a consistent distance from the camera.

The pre-test positioning verification process continues until the participant's face meets the specified criteria for proper alignment, as determined by the system's predefined thresholds and guidelines. Once the participant's face is correctly positioned, the system signals that the test can commence, automatically transitioning to the next phase of the HGN test. This seamless integration of face-api ensures that the HGN test is administered under standardized conditions, minimizing variability in eye gaze data and enhancing the reliability of the subsequent analysis performed by the machine learning model.

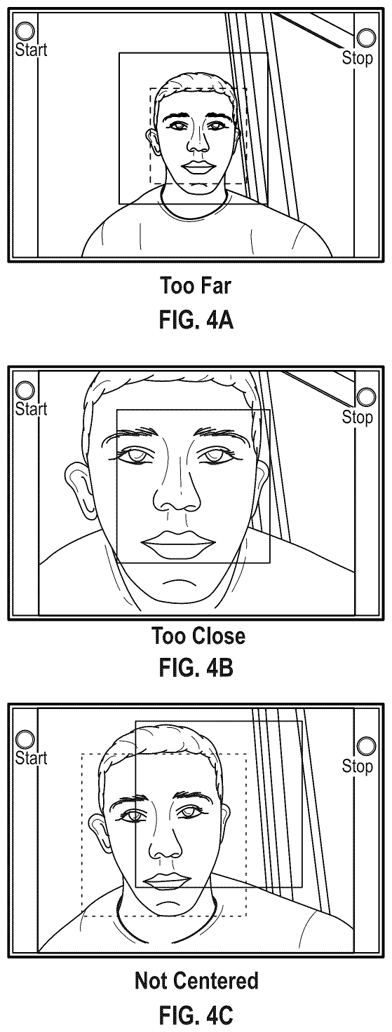

To achieve proper positioning, in accordance with an embodiment a python script that utilizes the OpenCV library and is supplemented by a Haar Cascades xml file, for example, may be used. This face recognition system places face recognition box around the user's face on the projection screen. In case the user's face is not in the ideal position for testing, visual cues may be displayed on the screen in the form of guiding squares of various colors and text in a graphical user interface to guide the user towards the correct positioning as shown in FIGS. 4A, 4B and 4C. FIG. 4A shows an example of a two guiding squares seen by the user when too far away from the screen; FIG. 4B illustrates a single guiding square shown the user in the GUI when too close to the screen; and FIG. 4C illustrates two guiding squares, which may be of different colors, to communicate when the user is not centered. Ideal positioning places the user's face at approximately 12 inches away from the camera and centered within the field of view for the camera. The proper location of the user's face is ensured by referencing the moving average of the X and Y coordinate pixels of the face recognition box. Likewise, the distance to the camera is determined by the moving average of the length and width pixels of the face recognition box. Once the HGN testing system has verified the user has positioned themselves correctly, the live feed disappears and the pretest face positioning stage is completed.

While a GUI has been shown in which guiding squares within the face recognition box are used to communicate to the user how to position within the face recognition box, the user interface need not be graphical and could instead be audio based with messages such as “move closer,” “move to the right,” “move to the left,” for example, to position the user correctly within the face recognition box displayed on the screen. This might be useful where a user has limited vision, for example.

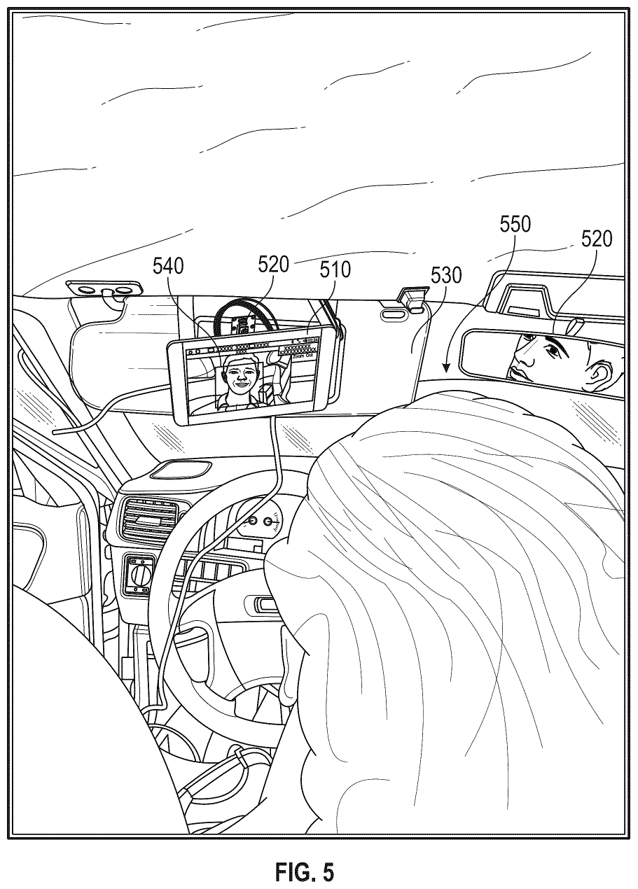

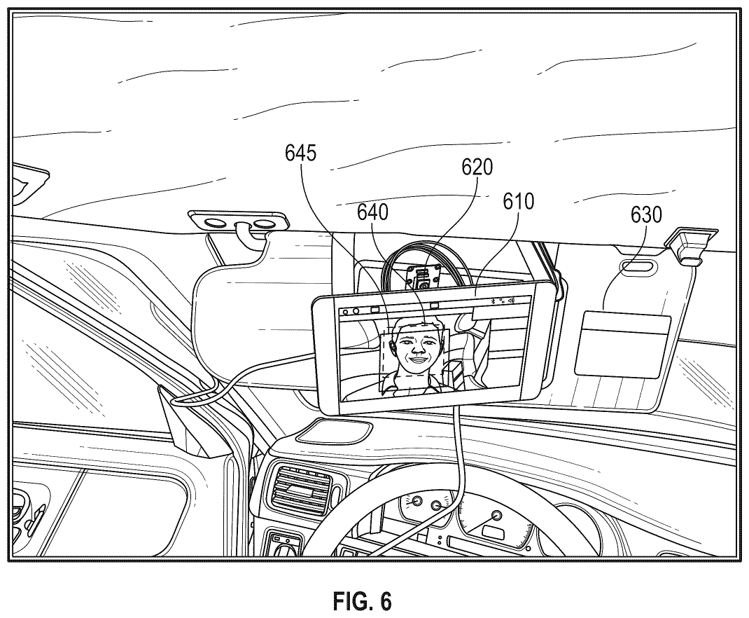

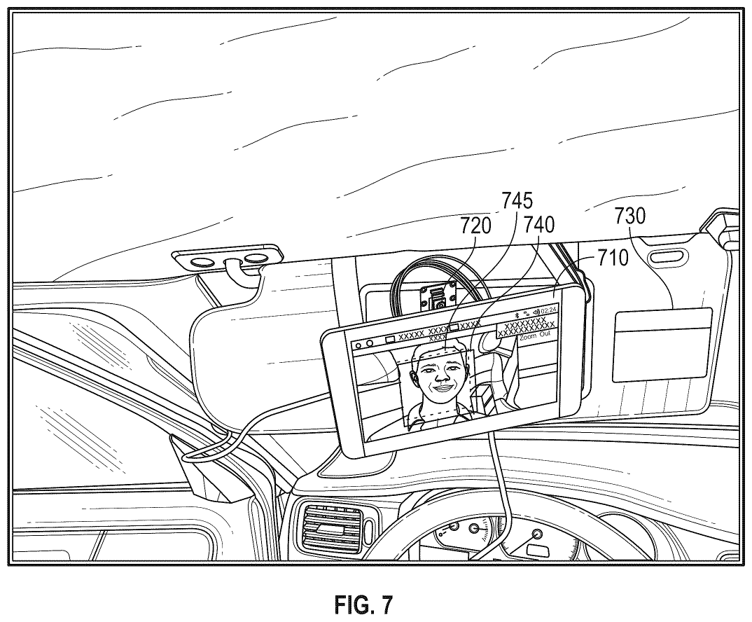

FIGS. 5, 6 and 7 further illustrate the pretest face positioning in the context of the HGN testing system in which the screen and image capture camera components of the HGN testing system in a vehicle are shown. In these drawings, placement of these components in or attached to a vehicle visor 530 is illustrated; in these examples, the screen 510 and camera 520 are attached to a preexisting visor but these components could also be integrated into one or more of the rearview mirror 520, heads-up display, dashboard, windshield, etc. of the vehicle. In FIG. 5, the screen 510 shows the user 550; the rearview mirror is also seen. A single guiding square 540 of a GUI within the screen is around the user's face. In FIG. 6, screen 610 and camera 620 are attached to a visor 630. Two guiding boxes 640, 645 are arrange around the image of the user on the screen with a message to “move closer” to the screen 610. In FIG. 7, screen 710 and camera 720 are attached to a visor 730. Two guiding boxes 740, 745 are arrange around the image of the user on the screen with a message advising that the user is “not centered” within the face recognition box in screen 710.

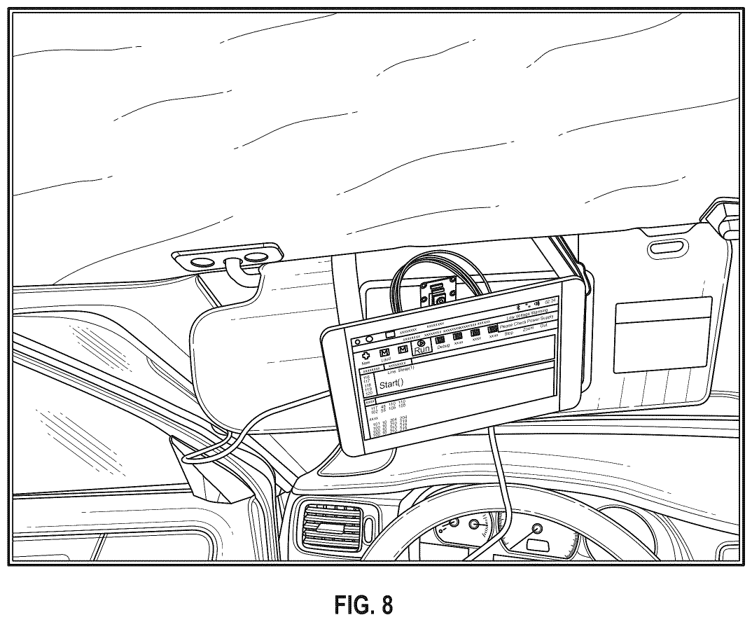

FIG. 8 illustrates a screen with a simulation test interface, showing “Start” and “Run” buttons on the screen.

Identification: The Identification stage is conducted in order to give users a personalized reference score, i.e. a reference HGN physiological state that is representative of a non-impaired state of a user, whenever the user completes a successful HGN simulation test attempt. A user's reference or baseline score is derived from their user profile's unique scores obtained during their training phase and a typical acceptable score among the average individual. If it is a user's first time attempting a test and they have not created a profile, then a separate one-time training phase will be executed. This training phase involves adding their face to the system's unique face recognition model and having the user complete 3 passing HGN tests to create their reference score. From there, a user can take a test and compare their current score to their reference score, giving them a sense of their current physiological state. This approach focuses on individualized analysis by comparing a user's current gaze behavior to their previously recorded baseline data. This method learns the distinctions between normal and impaired states on a per-user basis by evaluating the similarity between gaze patterns from different sessions. During real-time operation, the HGN testing system assesses how closely a new test aligns with the user's established non-impaired baseline. If a significant deviation is detected, it is used as an indicator of possible impairment.

Alternately, a user's reference score may not be specific to the user. This approach involves training a generalized model on a broad dataset composed of gaze tracking test results collected from a diverse population. These test results are labeled according to the test subject's physiological condition during testing, distinguishing between unimpaired and impaired states. By learning from a wide range of gaze patterns across various users and conditions, this approach enables the HGN testing system to identify common indicators of impairment based on generalized trends in eye movement behavior.

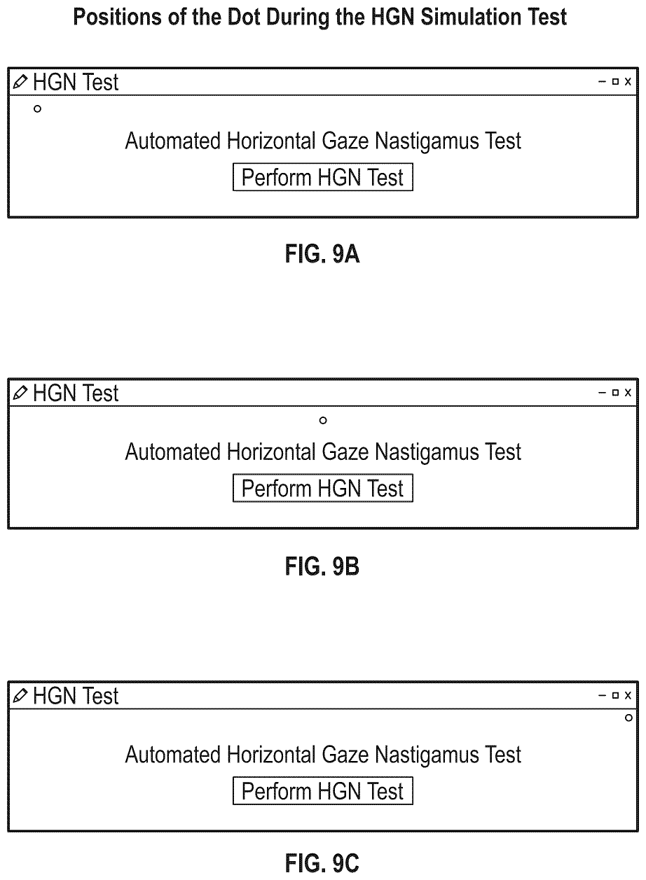

Visual Cue (Dot) Simulation: The visual cue simulation stage involves recording a video of the user's face while they participate in the HGN dot simulation test. This process may be accomplished using a Python script that accesses the connected webcam using the OpenCV library and projects a Graphical User Interface (GUI) using the tkinter library. With the standard screen being 12 inches in width and the user's face positioned 12 inches from the camera, the visual cue simulation achieves an angle that places the visual cue, shown here as a dot although other visual cues could be used, near the user's peripheral vision, activating a user's HGN. The test begins with a dot holding its position at the left edge of the screen for two seconds, after which it traverses horizontally across the screen for four seconds, until it reaches the right edge. The dot then holds its position for two more seconds until the test ends, after which the webcam stops recording and saves the video to memory. Different dot positions during an example HGN simulation test can be shown in FIGS. 9A, 9B, 9C. In FIG. 9A, the left position of the dot is shown at the left edge of the screen; FIG. 9B shows the dot sweeping from left to right across the screen, for example; FIG. 9C illustrates the right position of the dot at the right edge of the screen.

Head Movement Detection: The head movement detection stage ensures that the user's head stays positioned throughout the visual cue (dot) simulation, which is paramount for creating HGN and establishing accurate scoring. The solution involves analyzing the recorded video with a Python script, for example, and the same face recognition system as the pretest face positioning phase may be used. The script takes the size and position of the face recognition box over multiple frames throughout the video. The positioning coordinate values are then analyzed by their variance to determine if there was significant change among them. The variance value is then displayed to the user to reflect if their position was satisfactory during the testing phase.

Eye Gaze Tracking: The final stage is the eye gaze tracking stage where it is determined if the user exhibits HGN by scoring the facial landmarks of the user's gaze with a statistical model. The process starts by having the recorded video of the user's face during the test be processed by a feature extraction tool, such as OpenFace Feature Extraction.

OpenFace is an open-source facial behavior analysis toolkit that facilitates robust and efficient feature extraction from facial images. Developed by the Carnegie Mellon University Multimodal Communication and Machine Learning Laboratory, OpenFace offers a comprehensive set of tools for facial landmark detection, head pose estimation, facial action unit recognition, and eye gaze estimation. Leveraging deep neural networks, OpenFace excels in capturing intricate facial dynamics and subtle cues that are indicative of physiological impairment, such as those observed during HGN tests. By employing OpenFace in the analysis module of the controller, facial features and dynamics can be accurately extracted from individuals undergoing HGN tests, enabling their responses to be quantified and analyzed with precision. This feature extraction process serves as an important component in the HGN testing system's ability to assess a user's sobriety or impairment status effectively and confidentially, thereby enhancing its overall reliability and performance in promoting road safety for vehicles and safe operation for machines generally.

The OpenFace software analyzes each frame of the video and returns a spreadsheet of facial landmark values, including the X coordinate of each eyes' gaze. From there, determination of whether the user's eyes were tracking the visual cue is performed. For example, in an example model an OpenFace spreadsheet may be used to determine if the user's eyes were following the dot adequately.

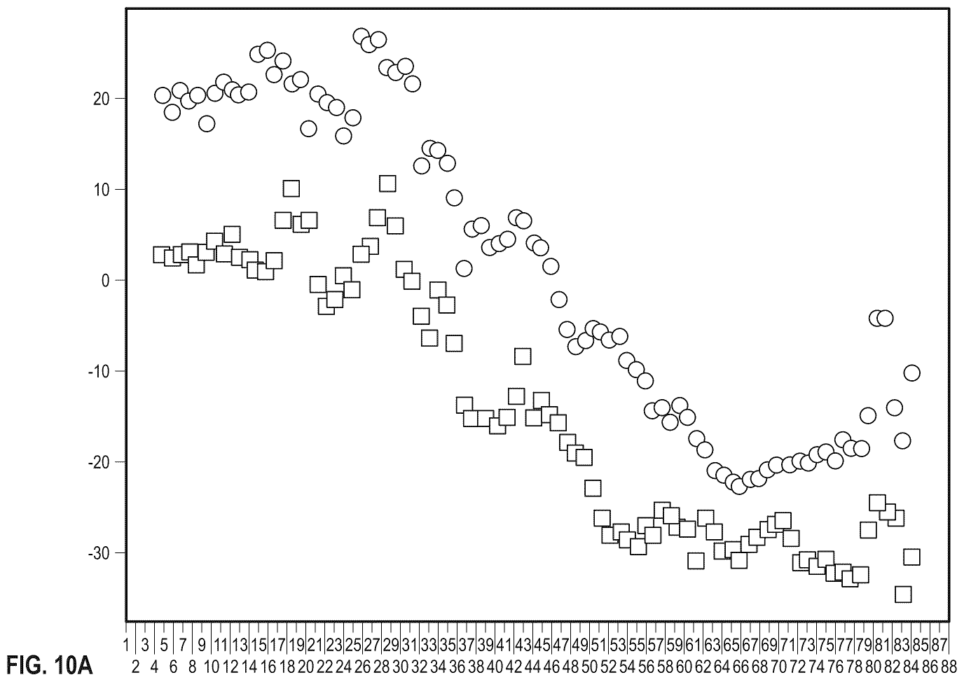

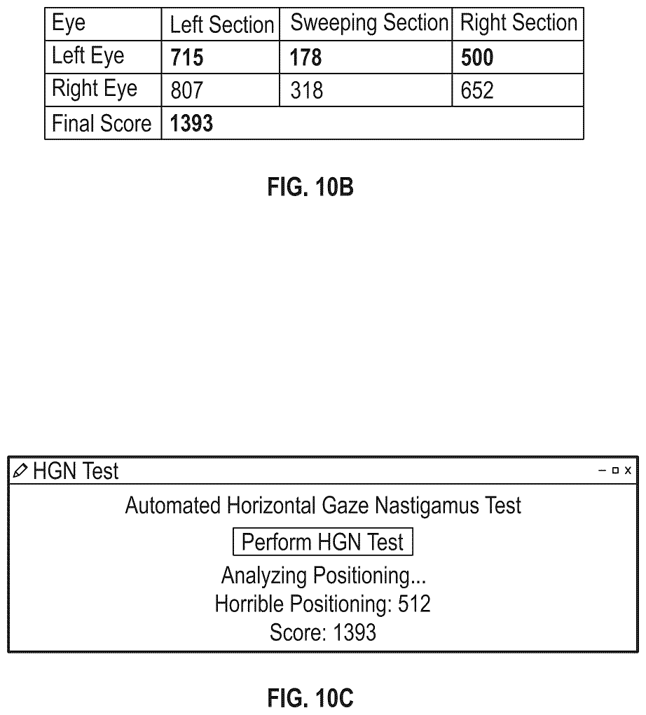

As an example, the overall model may account for the three separate sections during the dot simulation (left, sweeping, and right) and two eyes, so six separate univariate linear regression models are created. The linear regression line for each eye and section is made from the X coordinate gaze values found within the spreadsheet from OpenFace. From each of the linear regression models, a mean squared error is determined and the slope of the line recorded. If the slope of the models is not near zero for the stable sections or a negative slope for the sweeping sections, a failing score of 500 for the section is immediately returned. FIGS. 10A, 10B, 10C illustrate the results of analysis performed by an eye gaze tracking analysis module of a controller. FIG. 10A is a graph that illustrates scores for the left eye (the lower graph denoted by squares) and the right eye (the upper graph denoted by circles); FIG. 10B illustrates test scores for each eye for the left section, sweeping section and right section, as well as a final score; and FIG. 10C illustrates a message that might be displayed to the user via a GUI about the failed HGN simulation test. In the graph of FIG. 10A, the X axis represents the index of the frame in the video that is taken as the user performs the HGN test. The Y axis shows the X coordinate of the user's eye gaze. As the user looks left and right, the X coordinate increases and decreases, respectively. In the table of FIG. 10B, examples of a user's scores for each eye and the final score for a failed test are shown; once all three sections for both eyes are scored, the final score returned is the sum of the best scoring eye from each of the three sections.

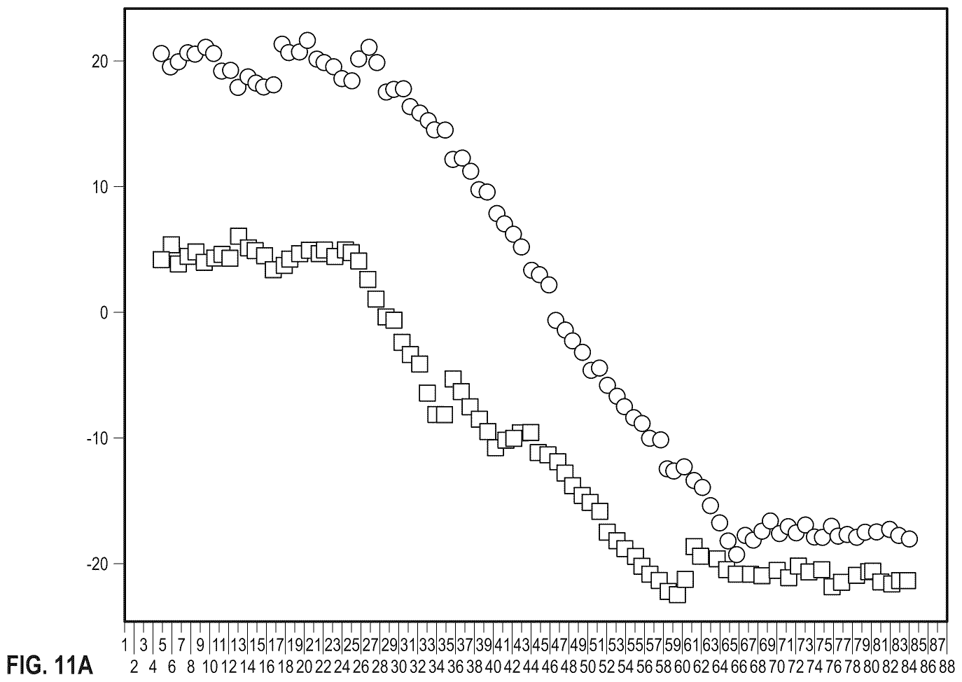

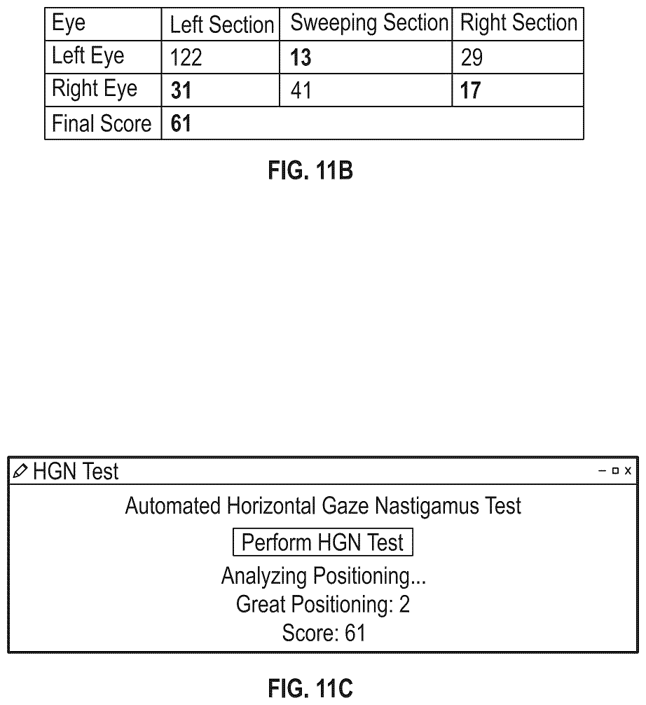

However, if the slope of the models is adequate and a passing score attained, the mean squared error is returned as the score as shown in FIGS. 11A-11C. FIG. 11A is a graph that illustrates scores for the left eye (the lower graph denoted by squares) and the right eye (the upper graph denoted by circles); FIG. 11B illustrates test scores for each eye for the left section, sweeping section and right section, as well as a final, passing score; and FIG. 11C illustrates a message that might be displayed to the user via a GUI about the passed HGN simulation test. In the graph of FIG. 11A, the X axis represents the index of the frame in the video that is taken as the user performs the HGN test. The Y axis shows the X coordinate of the user's eye gaze. As the user looks left and right, the X coordinate increases and decreases, respectively. In the table of FIG. 11B, examples of a user's scores for each eye and the final score for a passed test are shown; once all three sections for both eyes are scored, the final score returned is the sum of the best scoring eye from each of the three sections.

### Security and Privacy

An intention of the HGN simulation testing system is not to criminalize the users utilizing it or to make their data available as evidence of their driving habits, which could have adverse consequences, such as adversely impacting a driver's car insurance policy. Rather, the technology described herein gives drivers a means to assess their ability to drive safely. For these necessary security measures may be added to protect the user's data and privacy. All identifiable data collected to create user profiles or during an HGN may be encrypted using the secret-key cipher Advanced Encryption Standard in Galois Counter Mode (AES-GCM). Using the AES in GCM mode as, in addition to confidentiality, also ensures authentication and integrity of the data. Additionally, it is desirable to minimize the delay caused by the HGN test and AES authenticated encryption modes were shown to have minimal effect on the performance of applications running on lightweight platforms such as the RPi.

To create a user's profile in the HGN testing system, the capture element camera takes an adequate number of pictures, such as 150 pictures, of the user to ensure accurate identification. The user's pictures are converted into arrays of bytes and then encrypted used AES-GCM; the originals are destroyed. Every time an HGN simulation test is conducted, the user's identification data are decrypted and compared against the images gathered during the identification phase. This ensures that the baseline data for the correct user is loaded while protecting their privacy.

Within the HGN testing system, a profiler utilizing OpenCV and DeepFace libraries has been developed to manage user profiles effectively. When a participant's face is detected within the camera frame, the system swiftly compares it with existing profiles. If no match is found, signaling a new participant, an automated profile creation process ensues. This profile encapsulates fundamental facial features and attributes. As participants progress through subsequent tests, their facial characteristics undergo continual comparison with their profile. Upon a match, the system evaluates the participant's current condition against their average and typical results, particularly important in discerning impairment, such as intoxication levels.

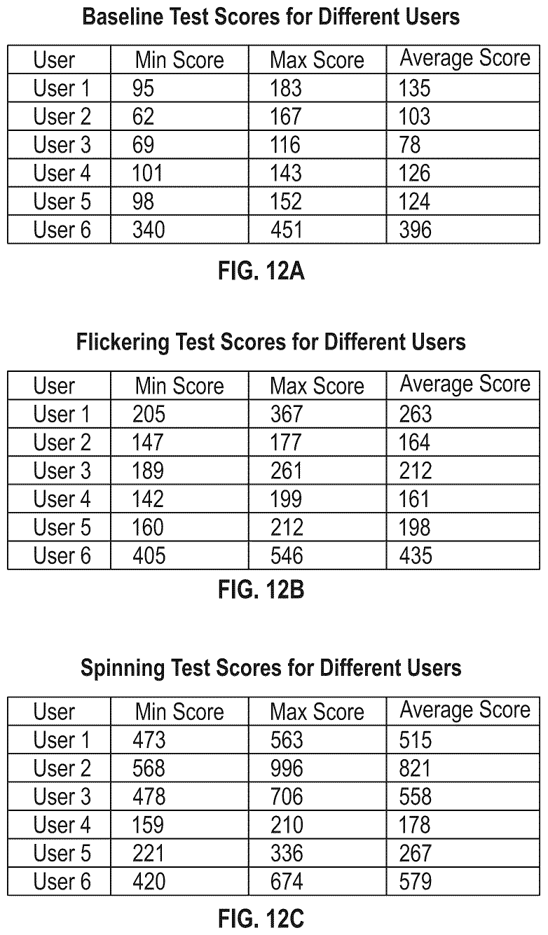

As an example, three different types of tests shown in FIGS. 12A, 12B, 12C were performed by six users to test the efficiency of the HGN testing system. The first set of tests established a baseline score for an individual where each user took the standard HGN test under normal sober conditions, see FIG. 12A. The next set of tests performed used a modified version of the standard HGN test, as shown in FIG. 12B. The modified version of the test only differed in the behavior of the visual cue dot during the simulation. When the dot was present on the screen, it would flicker left and right erratically, while still moving in the general movement of the original dot's path. With the user following the flickering location of the dot, this effect emulates nystagmus in a user's eye to further show that the HGN testing system can detect these minute eye movements. The last set of tests was performed using the standard HGN test. However, before users began each test, they were spun around for 30 seconds in order to simulate the disorientation of intoxication or other impairment such as drug use. Each of the three tests was conducted for each user 10 times and recorded the minimum, maximum and average score for each user as shown in FIGS. 12A-12C.

It can be shown from these test results for each user that there is a clear distinction between the scores in their normal, non-impaired state and when they are spun or following the flickering dot simulation tests. This distinction allows the HGN testing system to distinguish between a user's normal state and impaired state allowing it to send the correct signal to control operation of a machine, such as a digital signal to immobilize a vehicle, when needed.

During another study, each participant underwent a comprehensive testing regimen designed to evaluate the efficacy and accuracy of the HGN testing system under various conditions. Each participant recorded a total of thirteen HGN simulation tests, comprising five sober tests, five tests where participants deliberately induced erratic eye movements while tracking the dot's position, and three tests involving induced nystagmus. The five sober tests served as baseline measurements, allowing for the establishment of normal eye movement patterns and facilitating comparison with the next five failing test results. Furthermore, the final three tests involving induced nystagmus, achieved by spinning participants in a chair ten times, simulated conditions where nystagmus occurs naturally due to physiological factors such as alcohol intoxication or vestibular dysfunction. Notably, only three spinning tests were conducted as participants may develop increased tolerance or resistance to spinning effects with repeated exposure. By subjecting participants to these diverse testing scenarios, the study aimed to comprehensively evaluate the robustness and reliability of the HGN system in detecting and quantifying impairment across different contexts and conditions.

### An Example HGN Simulation Testing

In view of the above, the HGN simulation testing protocol may begin with a user removing any glasses they may be wearing or other impediments to viewing the screen to ensure optimal visibility and accuracy during the HGN test. Subsequently, users are prompted to initiate the test and position themselves correctly within the camera frame, guided by real-time visual feedback provided by the HGN testing system. As shown in FIG. 8, a user can interact with a GUI displayed on the screen to start and run the HGN simulation test. Once positioned correctly, discussed above, the test commences with the presentation of a visual cue, such as a dot stimulus, that moves horizontally across the screen. The dot starts at the left edge of the screen and gradually traverses to the right in a steady motion, pausing momentarily at the right edge before concluding the test. Throughout this process, users are instructed to maintain their gaze on the moving dot, allowing the system to capture and analyze their eye movements for signs of physiological impairment.

Upon completion of the test, the recorded video footage may be automatically transmitted to a backend server for storage and further processing. This standardized testing protocol ensures consistency and accuracy in assessing users' sobriety levels, facilitating reliable detection of impairment through the HGN test.

### Cloud Service Backend

For efficient and secure processing of user-generated videos and extraction of features for analysis, an example HGN system leverages a cloud-based backend infrastructure deployed on Amazon EC2 (Elastic Compute Cloud) servers. Upon completion of an HGN simulation test, the HGN testing system initiates the transfer of recorded video footage to the backend EC2 server, where it undergoes processing using the OpenFace facial behavior analysis toolkit. OpenFace is employed to extract relevant facial features and dynamics from the video, providing valuable data for subsequent analysis and classification of physiological impairment.

Following feature extraction, the resulting data, including the extracted features in CSV format and the original video file, may then be securely stored in an encrypted Amazon Simple Storage Service (S3) bucket. This encrypted storage ensures the confidentiality and integrity of the sensitive data, safeguarding it against unauthorized access or tampering. By utilizing Amazon S3's robust encryption mechanisms, stringent data privacy standards and compliance requirements are upheld, thereby fostering trust and confidence in the security of the HGN testing system.

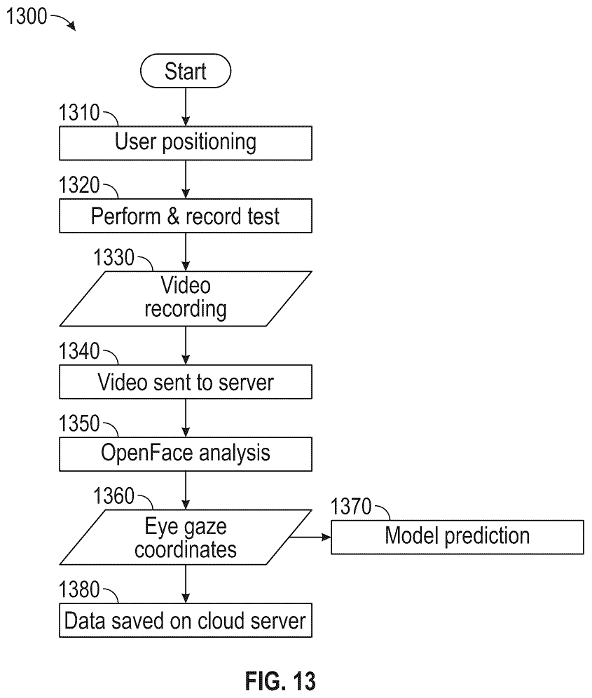

Referring to flow 1300 of FIG. 13, a HGN test flow representative of this process is presented. At 1310, the user or test subject is positioned for the HGN simulation test, within the face recognition box displayed on a screen and including maintaining proper head positioning of the test subject. At 1320, the HGN simulation test is performed and recorded. A video recording of the HGN date of the test subject is recorded at 1330. The video may be sent to a server or other storage means at 1340. An analysis of the HGN data of the test subject contained in the video is analyzed, such as by an analysis module of a controller, at 1350. Extracted eye gaze coordinates of the test subject from the analysis at 1360 are used to generate a model prediction at 1370. The data associated with the HGN test may be saved on a server, such as a cloud server, at 1380.

### Random Forest

Random Forest (RF) is a machine learning algorithm or model useful for its versatility and robustness in handling classification and regression tasks. It operates by constructing a multitude of decision trees during training and outputs the mode of the classes (classification) or mean prediction (regression) of the individual trees. Each decision tree is built on a randomly sampled subset of the training data and a random subset of features, ensuring diversity among the trees. When making predictions, RF aggregates the outputs of these trees, which collectively offer more accurate and stable results compared to a single decision tree. This approach mitigates the risk of overfitting while maintaining high predictive performance. The RF model plays an important role in processing the data obtained from the HGN tests. By leveraging RF, the HGN responses of individuals are effectively analyzed, compared to established baselines, and patterns indicative of impairment are discerned with a high degree of accuracy.

### Pre-Processing

Raw eye gaze coordinate data extracted from the frames of the video presents challenges in its direct applicability for classification tasks within the Random Forest model. Due to the inherent variability and noise present in the eye gaze coordinates across frames, direct utilization of this raw data can lead to suboptimal model performance. To address this issue, a preprocessing step aimed at extracting meaningful features from the raw eye gaze data may be implemented. Specifically, the data from each test sample is segmented into three distinct sections, reflecting different phases or intervals within the HGN simulation test. Both the mean squared error for each section and the summed absolute difference between each value and its neighbor, or intepoint distance, are then computed, quantifying the deviation of the eye gaze coordinates within that section. These processed values effectively encapsulate the variability and patterns present in the eye gaze behavior throughout the test duration, thereby serving as informative features for the Random Forest model. By employing this preprocessing strategy, the raw eye gaze data is transformed into a structured and interpretable format that enhances the discerning power of the model, ultimately enabling more accurate classification of physiological impairment based on HGN simulation responses.

### Machine Learning Model

To train and evaluate the Random Forest model for detecting impairment based on HGN responses, the testing data was transformed into a binary classification problem of pass and fail. Each participant's testing data was labeled as either 1 for passing or 0 for failing, with passing denoting sober tests and failing indicating induced erratic eye movements or induced nystagmus. The first five sober simulation tests recorded by each participant were assigned a label of 1, representing successful completion of the test under normal conditions. Conversely, the subsequent five simulation tests involving deliberately induced erratic eye movements and the three simulation tests involving induced nystagmus were labeled as 0, signifying impairment or abnormal eye behavior. This binary classification scheme enabled the Random Forest model to learn to distinguish between normal and impaired eye movement patterns, facilitating the accurate identification of impairment during the HGN simulation test. Training the model on this labeled dataset developed a robust and reliable classifier capable of effectively assessing a user's sobriety based on their HGN responses.

K Fold Validation: To properly assess the accuracy and generalization capability of the machine learning model, k-fold cross-validation with a value of k set to 10 was employed. This technique involves partitioning the dataset into k equal sized folds, where each fold serves as a validation set while the remaining k−1 folds are utilized for training. Subsequently, the model is trained and evaluated k times, with each fold taking turns as the validation set. Iteratively rotating the validation set across all folds ensures that every data point is used for validation exactly once, thereby minimizing the risk of bias and overfitting. Furthermore, the use of k-fold cross-validation provides a robust estimation of the model's performance metrics, including accuracy, precision, recall, and F1 score, across multiple iterations. The average performance metrics computed over the k iterations serve as reliable indicators of the model's effectiveness in discerning impairment based on the HGN responses. This rigorous validation approach confidently ascertains the model's accuracy and its ability to generalize to unseen data, thereby bolstering the credibility and reliability of systems for enhancing road and machine safety.

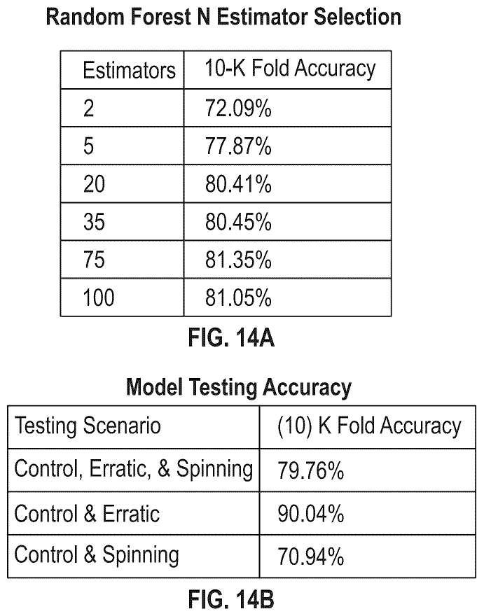

N Estimator Selection: After evaluating the performance of the Random Forest model across various numbers of estimators, the accuracy of the model stabilized at an estimator count of 75, as depicted in FIG. 14A.

### Model Accuracy

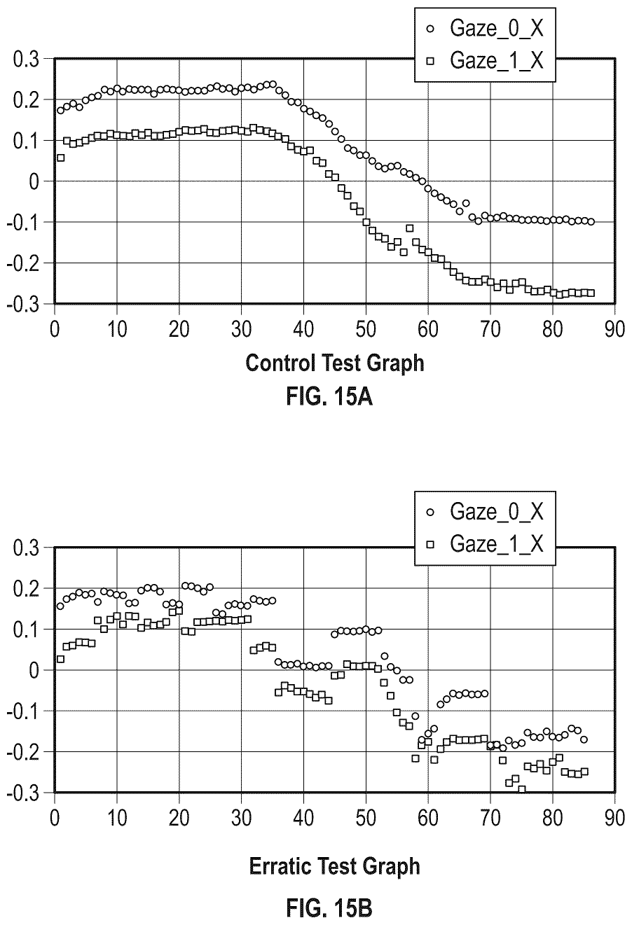

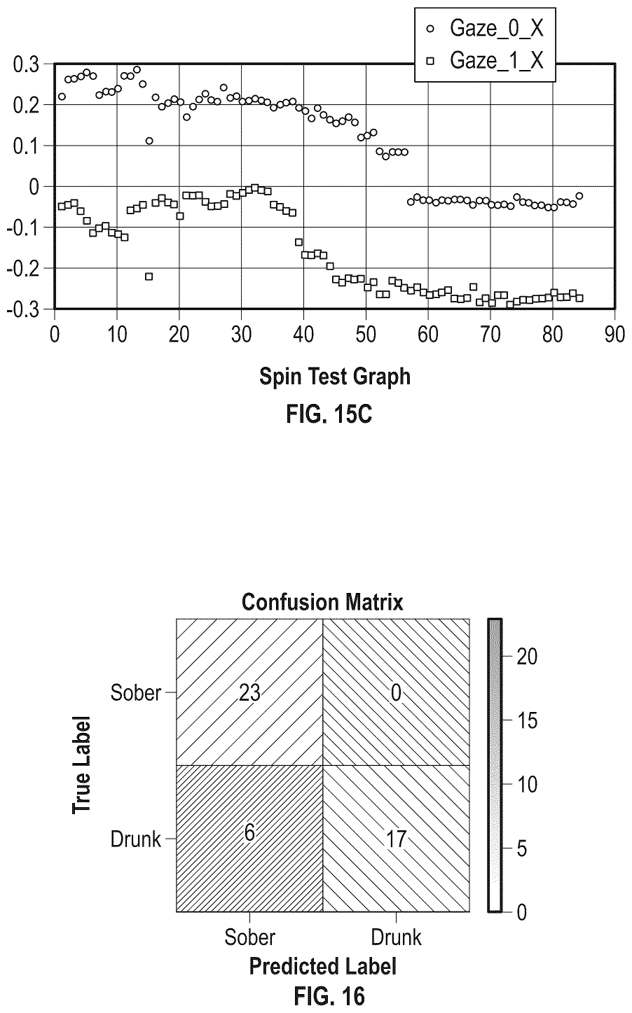

The results presented in FIG. 14B highlight the model's effectiveness in distinguishing between various testing scenarios. Particularly, the model performs robustly in identifying impairment during control tests where the subjects were asked track the dot visual cue normally and as accurately as possible, shown in the control test graph of FIG. 15A, as well as those with deliberately induced erratic eye movements that shift quickly from side to side of the dot or have trouble following it across the screen, achieving a high level of accuracy, illustrated in the erratic test graph of FIG. 15B. However, when faced with the added complexity of dizziness induced nystagmus generated by spinning the test subject quickly in a chair, illustrated in the spin test graph of FIG. 15C, the model's accuracy diminishes, indicating some challenges in accurately identifying impairment under these conditions. Despite this, the model still demonstrates a notable capability to differentiate between impaired and non-impaired states across diverse testing conditions, emphasizing its potential value in enhancing road safety through early detection of impairment.

FIG. 15A is a test graph illustration of a passed test. FIG. 15B is an illustration of an erratic test, taken from a deliberately induced erratic eye movements of a test subject. The erratic test graph may be performed by asking the test subject to deliberately delay their gaze when tracking the dot, i.e. not being able to smoothly follow it. The approach approximates the nystagmus found in test subjects impaired by alcohol, for example. FIG. 15C illustrates a spin test graph taken after a test subject is spun a number of times, such as ten times. In each of these graphs of FIGS. 15A, 15B, 15C, the X axis of the graphs represents the index of the frame in the video that is taken as the test subject performs the HGN test. The Y axis of the graphs shows the X coordinate of the test subject's eye gaze. As the subject looks left and right, the X coordinate increases and decreases, respectively. The upper graph (in circles) and the lower graph (in squares) in each drawing represents the left and right eyes, respectively, of a test subject.

### Preventing False Positives

The HGN testing system aims to cut down the number of drunk driving fatalities and other problems caused by operation of machinery while impaired. However, the base model has a chance to impede users who are sober or non-impaired. Achieving 100 percent accuracy in preventing false positives, where individuals who are sober and non-impaired do not fail the test, requires a nuanced approach that accounts for the intricacies of the HGN responses and the underlying features driving impairment detection. One effective strategy employed involves refining the model's decision boundary to maximize specificity without sacrificing sensitivity. By adjusting the threshold for classifying responses as impaired, the likelihood of misclassifying sober individuals is minimized while maintaining the ability to reasonably identify impairment when present. Reference is made to the tailored model confusion matrix of FIG. 16.

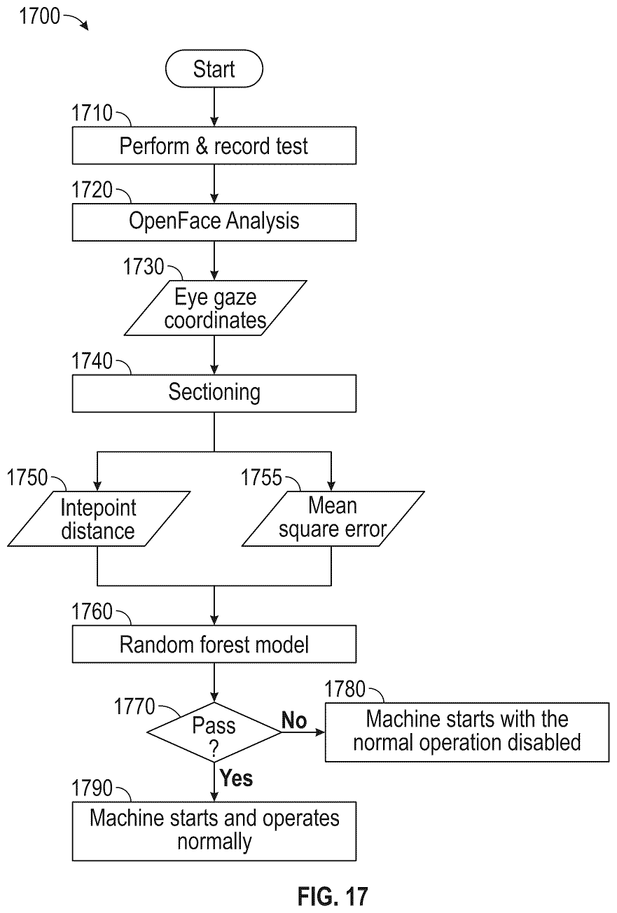

In view of the foregoing and referring now to FIG. 17, a flow 1700 representative of HGN data processing is shown. At 1710, the HGN simulation test is performed and recorded, followed by analysis, such as OpenFace analysis, of the captured HGN data of the test subject at 1720. Eye gaze coordinates of the test subject are obtained at 1730 and sectioning of the eye gaze coordinates occurs at 1740. Both the mean squared error for each section and the intepoint distance, i.e. the summed absolute difference between each value and its neighbor, are then computed, quantifying the deviation of the eye gaze coordinates within that section, at 1750 and 1755. These values are used by a personalized classification module, such as a random forest (RF) machine learning algorithm or a Siamese neural network, to determine whether the test subject has passed or failed the HGN simulation test at 1760. If the test subject has failed the HGN simulation test, normal operation of a machine disabled at 1780. In the example of a vehicle, this may include disablement of mobilization element of the vehicle, thereby circumventing or preventing operation of the vehicle by the test subject. If the test subject passes the HGN simulation test, the machine starts and operates normally for the test subject at 1790. In the case of a driver of a vehicle, the driver is able to normally start and drive the vehicle.

### Machine Modification/Control Responsive to Failed HGN Simulation Test

In accordance with various embodiments of the disclosure, a HGN simulation test is used to determine a user's impairment state of a machine, such as a vehicle or other machine, and that the HGN testing system is a power technology providing protections from the dangers of operating machinery while impaired. The HGN testing system is unique in that for certain applications, such as vehicles, it need not prevent the machine from starting but only immobilize it while making all functionalities such as heating, cooling and charging available to the vehicle riders. Reference is again made to flow 300 of FIG. 3.

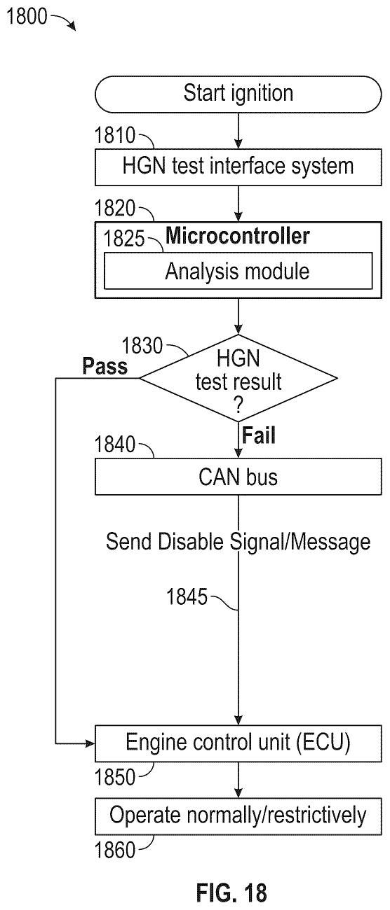

More particularly, in accordance with various embodiments of the disclosure, a HGN simulation test is used to prevent operation of a vehicle by an impaired, e.g. drugged, drunk or otherwise impaired, driver. Referring now to the functional block diagram 1800 of FIG. 18, a driver of the vehicle interfaces with the HGN simulation test through a HGN test interface system 1810, having a user interface for so doing. The interface and operation of the HGN simulation test is controlled by a controller, such as microcontroller 1820, which controls operation of the test via the HGN test interface system 1810 and an analysis conducted by an analysis module 1825 of microcontroller 1820. The analysis and data of the analysis module 1825 are used to determine the current HGN physiological state of the driver, by comparison with a reference HGN physiological state. If it is determined at Decision Block 1830 that the current HGN physiological state of the driver is outside an acceptable range, such as a test score that falls below a reference test score, then the driver fails the HGN simulation test and indication of the failure, such as a digital signal, 0 or 1, is sent to the Controller Area Network (CAN) of the vehicle, an in-vehicle bus network that allows microcontrollers and the Engine Control Unit (ECU) 1850 to communicate without the need for a host computer and accordingly may reside in the Cloud, acceptable through wireless communication means. The CAN bus 1840 sends a disable signal 1845, such as a digital signal or a disable message, to the ECU 1850, which responsively disables or prevent mobilization of the vehicle. If, however, the driver passes the HGN simulation test a pass signal or message is provided to 1850. The vehicle operates normally or restrictively at 1860 in accordance with whether ECU 1850 receives a pass signal/message or a fail signal/message.

With respect to the disable signal or message 1845 send by the CAN bus 1840 to ECU 1850, there are many ways to at least immobile operation or movement of a vehicle, whether the vehicle is an automobile, a bus, a taxi, a delivery service, a boat, ship or ferry, a train, a heavy operating vehicle such as a large truck, crane or forklift, etc. All such vehicles potentially have need to restrain mobilization of the vehicle to only non-impaired drivers. By way of example and not limitation, disable signal/message 1845 could cause any of the following, singly or in combination: disable shifting; zero out the throttle request; disable fuel injectors; force engage the Electric Parking Brake (EPB); and only allow accessory mode and engine cranking.

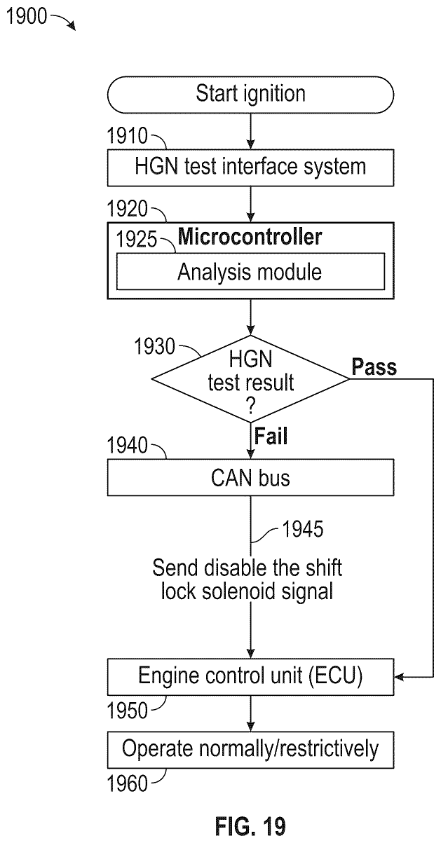

Referring to the functional block diagram 1900 of FIG. 19, a driver of the vehicle interfaces with the HGN simulation test through a HGN test interface system 1910. The interface and operation of the HGN simulation test is controlled by a controller, such as microcontroller 1920, which controls operation of the test via the HGN test interface system 1910 and an analysis conducted by an analysis module 1925 of microcontroller 1920. The analysis and data of the analysis module 1925 are used to determine the current HGN physiological state of the driver, by comparison with a reference HGN physiological state. If it is determined at Decision Block 1990 that the current HGN physiological state of the driver is outside an acceptable range, such as a test score that falls below a reference test score, then the driver fails the HGN simulation test and indication of the failure, such as a digital signal, is sent to the Controller Area Network (CAN) 1940 of the vehicle. In this example, CAN bus 1940 sends a disable signal 1945 to the ECU 1950 to disable the shift lock solenoid signal, thereby responsively disabling or preventing mobilization of the vehicle. If, however, the driver passes the HGN simulation test a pass signal or message is provided to 1950. The vehicle operates normally or restrictively at 1960 in accordance with whether ECU 1950 receives a pass signal/message or a fail signal/message.

Such signaling provides a transmission interlock system by which a vehicle is immobilized; this is believed unique to the HGN testing system presented herein. This feature allows intoxicated or impaired individuals to use life-saving features in the vehicle, such as electricity, temperature control, etc., while inside their vehicle. This is because the test begins upon the start of the engine, yet the car will not be removed from park (the lowest gear), until the sobriety test is passed.

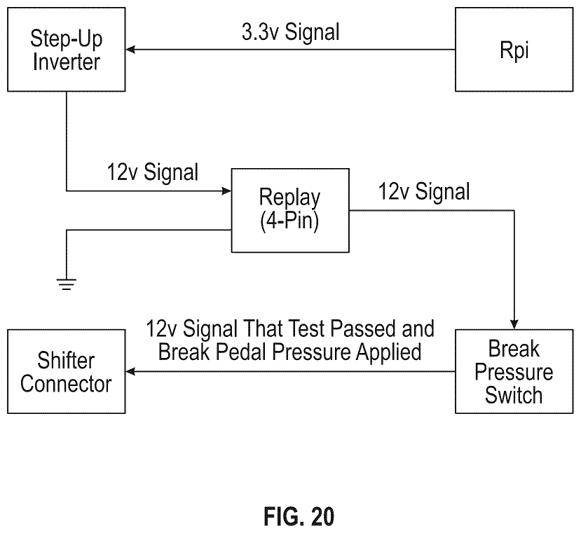

In accomplish any of these immobilization commands, wiring diagrams of the engine control unit (ECU) published by the vehicle manufacturer can be followed to determine what, where and when such immobilization signals and message are needed for a given vehicle. Consider the example of finding the brake pressure switch to disable the shifting mechanism. The brake pressure switch sends a signal to the vehicle's computer controller, indicating that the brake pedal has been depressed. In a vehicle with an automatic transmission, a driver is not able to shift a vehicle out of the parking gear without pressing the brake pedal. Using a digital multi-meter (DMM) and testing for resistance between various points on the wires to identify and label them, as well as to trace the wires through the vehicle. This allows determination of where to install the HGN testing system/device so that it results in the correct response when the test is complete. By adding the HGN testing system and a relay in this location, control of the switch can be activated or not based upon the driver's HGN simulation test results. If the driver passes the HGN simulation test, then a signal 1845, 1945 is sent to the relay, closing the circuit, allowing for the brake pressure switch to be activated. Once the brake pressure switch is activated, the car is able to move from park. If the driver fails the HGN simulation test, the switch does not receive the necessary voltage to close the opened circuit thereby immobilizing the vehicle. An example immobilizing system is illustrated in FIG. 20.

In accordance with various embodiments of the disclosure, a passive transmission interlock system utilizes the HGN simulation test to determine a driver's impairment state. The HGN testing system is unique in that it does not prevent the vehicle from starting but only immobilizes it making all functionalities such as heating, cooling and charging available to the vehicle riders. The test results show that the HGN testing system and methodology can identify an impairment state for different users.

The HGN system and methodology of the present disclosure is different from other solutions for a variety of important reasons. The solution is passive, not requiring much effort from the user; it need not prevent the machine or vehicle from starting for a failed test, and it does not have the issues associated with devices that require certain temperatures to function properly, such as is the case with breathalyzers.

As previously described, the most pervasive form of sobriety testing is via a breathalyzer, which may also be used to prevent vehicles from being started, should the driver fail the test. As such, the breathalyzer-activated vehicle ignition system is a primary alternative for the technology presented herein. That said, certain competitive advantages are provided by the technology of the present disclosure:

HGN is not affected by certain medications or medical conditions. Breathalyzers measure blood alcohol content (BAC), which can be affected by certain medications, such as cold medicine, and medical conditions, such as diabetes. HGN is not affected by these factors, so it can be used to screen drivers who may be impaired even if they have taken medication or have a medical condition.

HGN is less susceptible to cheating. Breathalyzers can be easily cheated by drivers who hold their breath or blow into the device slowly. HGN is more difficult to cheat, as it requires the driver to follow the instructions programmed into the HGN simulation test and keep their eyes still.

HGN can be used to screen drivers for other types of impairment. Breathalyzers only measure BAC, so cannot be used to screen drivers for impairment caused by drugs other than alcohol. HGN can be additionally used to screen drivers or machine operators for impairment caused by drugs, fatigue, or other factors.

Embodiments of the invention have been described to explain the nature of the invention. Those skilled in the art may make changes in the details, materials, steps and arrangement of the described embodiments within the principle and scope of the invention, as expressed in the appended claims.

While implementations of the disclosure are susceptible to embodiment in many different forms, there is shown in the drawings and will herein be described in detail specific embodiments, with the understanding that the present disclosure is to be considered as an example of the principles of the disclosure and not intended to limit the disclosure to the specific embodiments shown and described. In the description above, like reference numerals may be used to describe the same, similar or corresponding parts in the several views of the drawings.

In this document, relational terms such as first and second, top and bottom, and the like may be used solely to distinguish one entity or action from another entity or action without necessarily requiring or implying any actual such relationship or order between such entities or actions. The terms “comprises,” “comprising,” “includes,” “including,” “has,” “having,” or any other variations thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Reference throughout this document to “one embodiment,” “certain embodiments,” “an embodiment,” “implementation(s),” “aspect(s),” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C.” An exception to this definition will occur only when a combination of elements, functions, steps or acts are in some way inherently mutually exclusive. Also, grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. References to items in the singular should be understood to include items in the plural, and vice versa, unless explicitly stated otherwise or clear from the text.

Recitation of ranges of values herein are not intended to be limiting, referring instead individually to any and all values falling within the range, unless otherwise indicated, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” “for example,” or the like) provided herein, is intended merely to better illuminate the embodiments and does not pose a limitation on the scope of the embodiments. No language in the specification should be construed as indicating any unclaimed element as essential to the practice of the embodiments.

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described. The description is not to be considered as limited to the scope of the embodiments described herein.

In the following description, it is understood that terms such as “first,” “second,” “top,” “bottom,” “up,” “down,” “above,” “below,” and the like, are words of convenience and are not to be construed as limiting terms. Also, the terms apparatus, device, system, etc. may be used interchangeably in this text.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art. it is not desired to limit the disclosure to the exact construction and operation illustrated and described, and, accordingly, all suitable modifications and equivalents may be resorted to that fall within the scope of the disclosure.

What is claimed is:

1. A system for horizontal gaze nystagmus (HGN) testing, comprising:
a controller having an analysis module;
a screen in communication with and controlled by the controller, the screen operable to display a face recognition box to a user and a user interface viewable by the user operable configured to communicate with the user to position the user within the face recognition box, as controlled by the controller; and
a capture element in communication with the screen and controlled by the controller;
where the controller controls the screen and the capture element to conduct a HGN simulation test of a user prior to the user taking an action that is verboten in an impaired state, including:
the user interface is configured to dynamically position the user within the face recognition box of the screen within an acceptable distance of the capture element;
perform a simulation test of the user positioned within the face recognition box during which the user follows a visual cue displayed on the screen and the visual cue traversing horizontally from a first edge of the screen to a second edge of the screen and capturing HGN eye movements of the user;
the analysis module of the controller is configured to analyze the captured HGN eye movements of the user to determine a current HGN physiological state of the user present during the simulation test, the current HGN physiological state indicated by the captured HGN eye movements; and
compare the current HGN physiological state of the user to a reference HGN physiological state representative of a non-impaired state of the user,
when the current HGN physiological state of the user falls outside an acceptable range of the reference HGN physiological state, indicate that the current HGN physiological state of the user is outside the acceptable range and impaired.

2. The system of claim 1, where the user interface is configured to position the user within the face recognition box as determined by a moving average of the length and width pixels of the face recognition box.

3. The system of claim 1, where the user interface is one or more of a graphical user interface and a user interface using audio to communicate with the user.

4. The system of claim 1, where the analysis module of the controller is configured to extract from the captured HGN eye movements of the user one or more facial features of the user and analyze the extracted one or more facial features of the user to determine the HGN physiological state of the user during the simulation test.

5. The system of claim 4, where said analysis module of the controller is configured to extract and preprocess data representative of the captured HGN eye movements, including to segment the data into two or more sections and for each section determine a deviation of eye gaze coordinates.

6. The system of claim 5, where the deviation of eye gaze coordinates within a section is derived from a mean squared error and a summed absolute difference between the section and an adjacent section of the two or more sections.

7. The system of claim 6, the data representative of the captured HGN eye movements including raw eye gaze data of the user.

8. The system of claim 1, where the reference HGN physiological state is determined by a non-impaired, baseline HGN physiological state specific to the user and further where the controller controls the screen and the capture element to generate the baseline HGN physiological state of the user by performing a baseline simulation test of the user and capture HGN eye movements of the user during the baseline simulation test.

9. The system of claim 8, where the analysis module of the controller is configured to derive a reference score of the user from one or more training simulation tests during a training phase of the baseline simulation test of the user and where comparison of the current HGN physiological state of the user to the reference HGN physiological state includes the analysis module of the controller comparing a current score of the user to the reference score of the user, the current score generated by analyzing the captured HGN eye movements of the user.

10. The system of claim 9, where the analysis module of the controller is further configured to encrypt data representative of the captured HGN eye movements of the user during the baseline simulation test and the reference score of the user.

11. The system of claim 9, where during the training phase the analysis module further configured to train on data representative of the captured HGN eye movements of the user using a personalized classification model.

12. The system of claim 11, where the personalized classification module is one or more of a random forest (RF) machine learning algorithm and a Siamese neural network.

13. The system of claim 1, where the analysis module of the controller is configured to compare decrypted data representative of reference HGN eye movements of the HGN physiological state of the user to the current HGN physiological state of the user.

14. The system of claim 1, where the system is a machine system and the controller is a controller of the machine, the controller configured to control operation of a machine, including:
the user interface configured to dynamically position the user within the face recognition box of the screen within an acceptable distance of the capture element, the screen and the capture element coupled to the machine;
perform the simulation test of the user positioned within the face recognition box;
the analysis module of the controller configured to analyze the captured HGN eye movements of the user to determine the current HGN physiological state of the user present during the simulation test;
compare the current HGN physiological state of the user to the reference HGN physiological state representative of the non-impaired state of the user; and
responsive to the current HGN physiological state of the user falling outside the acceptable range of the reference HGN physiological state, generate a failure signal and responsive to the failure signal the machine temporarily restricting operation of the machine by the user.

15. The system of claim 14, further including the machine may be turned on before the user is dynamically positioned, before the simulation test is performed, or after the current HGN physiological state of the user is compared to the reference HGN physiological state when the current HGN physiological state of the user does not fall outside the acceptable range of the reference HGN physiological state.

16. The system of claim 14, where one or more of the screen and the capture element are coupled to one or more of a visor, a rearview mirror, a heads-up display, a windshield and a dashboard of the machine or integrated with one or more of the visor, the rearview mirror, the heads-up display, the windshield and the dashboard of the machine.

17. The system of claim 14, where the system is a vehicular system having a controller area network (CAN) bus and an Engine Control Unit (ECU), the CAN and the ECU in cooperative communication to control mobilization of the vehicle.

18. The system of claim 17, where when the current HGN physiological state of the user falls outside an acceptable range of the reference HGN physiological state, the analysis module generates a failure signal is provided by the CAN bus to the ECU that temporarily prevent the user from mobilizing the vehicle responsive to receipt of the failure signal.

19. The system of claim 18, where responsive to generation of the failure signal, the controller controls one or more of gears, transmission, and brake pressure switch of the vehicle to immobilize the vehicle.

20. The system of claim 18, where generation of the failure signal further includes the analysis module generates a digital signal used by the ECU to control an interlock component of the vehicle and temporarily immobilize the vehicle.
